(19) United States
(12) Patent Application Publication
(10) Pub. No.: US 2021/0162163 A1
(43) Pub. Date: Jun. 3, 2021

(54) SLEEP SUITE

(71) Applicant: David STEER, St. Albert (CA)

(72) Inventor: David STEER, St. Albert (CA)

(21) Appl. No.: 17/170,341

(22) Filed: Feb. 8, 2021

Related U.S. Application Data
(63) Continuation of application No. PCT/CA2019/051078, filed on Aug. 6, 2019.
(60) Provisional application No. 62/714,891, filed on Aug. 6, 2018.

(51) Int. Cl. A61M 21/00; H04W 4/38; A61M 21/02; A47C 21/00; A47C 21/04; G05B 19/042

(52) U.S. Cl. CPC A61M 21/0094; G05B 2219/2614; A61M 21/02; A47C 21/003; A47C 21/042; G05B 19/0426; A61M 2021/0016; A61M 2021/0027; A61M 2021/0044; A61M 2021/0066; A61M 2205/3313; A61M 2205/3368; A61M 2205/3375; A61M 2230/10; A61M 2205/3584; H04W 4/38

(57) ABSTRACT

A sleep suite includes a main structure defining a sleep chamber, a plurality of sensors configured to monitor conditions of the sleep chamber, a plurality of devices configured to change the conditions of the sleep chamber, and a control panel. The control panel automatically controls the plurality of devices to change the conditions of the sleep chamber based on predetermined settings for the plurality of devices and the conditions monitored by the plurality of sensors.

### CROSS-REFERENCE TO RELATED APPLICATIONS

This application is a continuation of International Application No. PCT/CA2019/051078, filed on Aug. 6, 2019, which claims priority to and the benefit of U.S. Provisional Application No. 62/714,891, titled “SLEEP SUITE” and filed Aug. 6, 2018. The disclosures of the above applications are incorporated herein by reference.

### FIELD

The present disclosure relates to enclosed chambers, and more particularly to a sleeping chamber in which humans may sleep.

### BACKGROUND

The statements in this section merely provide background information related to the present disclosure and may not constitute prior art.

Various chambers for sleeping individuals have been designed. The chambers may not have room conditions tailored for a particular user. The chambers may be limited to certain functions. For example, the chambers may be designed for sleep only. These issues associated with sleeping chambers are addressed by the present disclosure.

### SUMMARY

In one form, a sleep suite is provided, which includes a main structure defining a sleep chamber, a plurality of sensors for monitoring conditions of the sleep chamber, a plurality of devices for changing the conditions of the sleep chamber, and a control panel. The control panel automatically controls the plurality of devices to change the conditions of the sleep chamber based on predetermined settings for the plurality of devices and the conditions monitored by the plurality of sensors.

In other features, the plurality of devices include at least one of a noise cancellation device, a lighting device, an aromatherapy device, a speaker, a fan, a chromotherapy device, a humidifier, a dehumidifier, an air filter, a ventilation device, and a water filter. The plurality of sensors include at least one of temperature sensor, a humidity sensor, an audio sensor, an aroma sensor, a motion sensor, an infrared sensor, an insect detector, a weight sensor, a dust sensor, a light sensor, a gas sensor, and a brainwave sensor. The plurality of sensors are arranged in an array throughout the sleep suite.

The sleep suite may further include a computer connected to the plurality of sensors, the computer configured to process and analyze information acquired by the plurality of sensors and related to the conditions of the sleep chamber. Alternatively, instead of a computer, the plurality of sensors may be in communication with a cloud server. The information acquired by the plurality of sensors may be processed and analyzed by a cloud server. The control panel is configured to access data relating to the predetermined settings of the plurality of devices from a group consisting of a computer, a hand-held device, a network server, and a cloud server.

The control panel may be configured to be activated by at least one of voice, passcodes, or key FOB. The control panel may further include at least one of a thumb print reader and a face reader.

The control panel includes a connection interface configured to connect to a hand-held device. The hand-held device stores settings for the plurality of devices. The control panel controls the plurality of devices simultaneously according to the settings stored in the hand-held device when the hand-held device is connected to the connection interface.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

The present disclosure will become more fully understood from the detailed description and the accompanying drawings, wherein:

FIG. 1 is a schematic view of a sleep suite constructed in accordance with the teachings of the present disclosure;

FIG. 2 is a schematic view of the sleep suite of FIG. 1, showing an arrangement of a plurality of sensors in the sleep suite;

FIG. 3 is a schematic view of a control panel, a plurality of devices, and a plurality of sensors disposed in a sleep suite constructed in accordance with the teachings of the present disclosure;

FIG. 4 is a schematic view showing a communication between a control interface and a plurality of devices; and

FIG. 5 is a schematic view showing a communication between a control interface and a plurality of sensors.

Corresponding reference numerals indicate corresponding parts throughout the several views of the drawings.

### DETAILED DESCRIPTION

The following description is merely exemplary in nature and is not intended to limit the present disclosure, application, or uses.

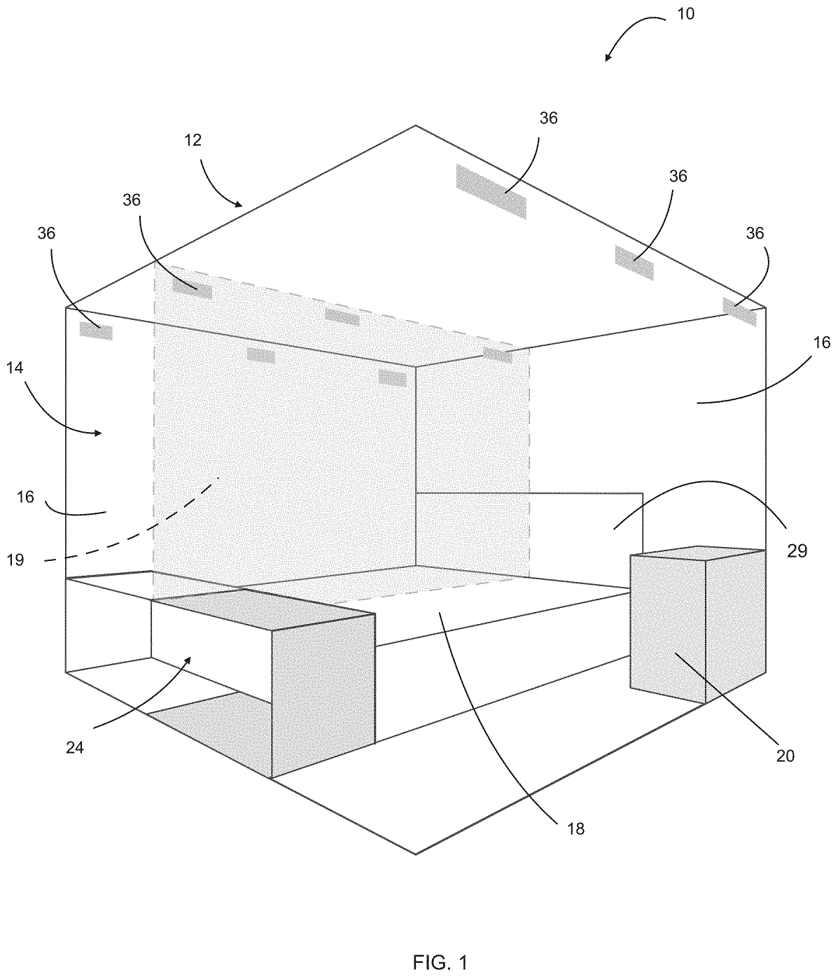

Referring to FIG. 1, a sleep suite 10 constructed in accordance with the teachings of the present disclosure includes a main structure 12 defining a sleeping chamber 14. The main structure 12 may include rigid or soft walls 16 and may be made of a material selected from a group consisting of wood, composite, glass, drywall and metal, intelligent/dynamic surface, and fabric. In one form, one or more of the walls 16 of the main structure 12 may use an existing wall or floor, such as a floor or a wall of a room or a divider wall 19 or a limo wall (shown in dashed lines in FIG. 1) of a motor vehicle. The main structure 12 may include an access door (not shown) to allow access into the sleeping chamber 14. The access door may be, for example, a hinged door, a zipper fabric door, a sliding door, or a clam shell door, among others.

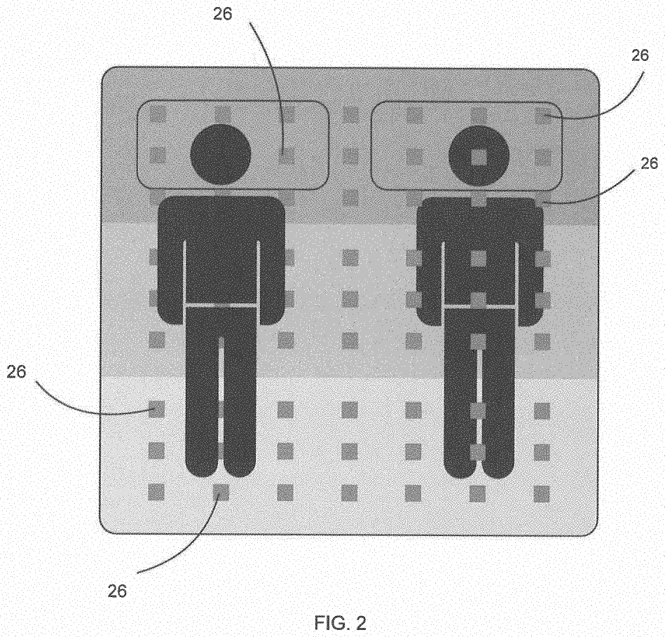

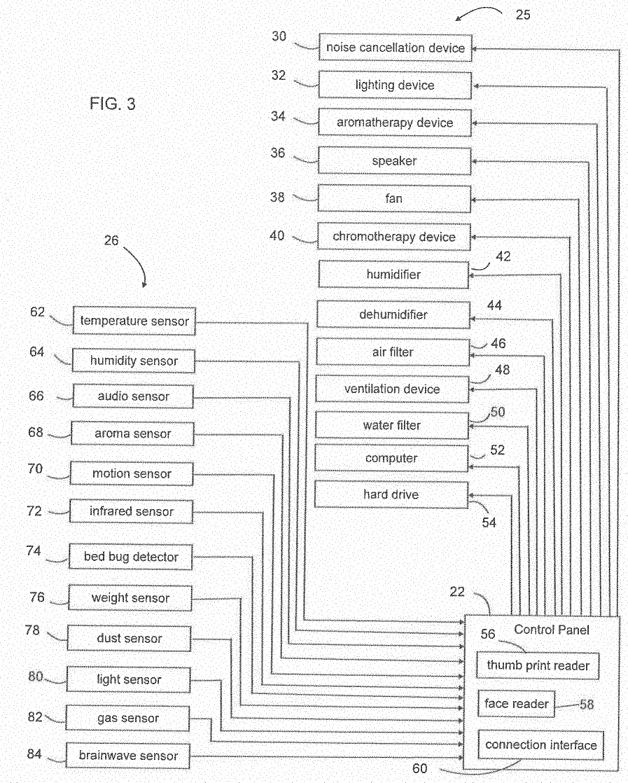

The sleep suite 10 further includes a mattress or a sleeping surface 18, on which a blanket and pillow set (not shown) may be disposed, a cabinet 20 (or a side table), a control panel 22 (shown in FIG. 3) mounted on the cabinet 20, a foot board 24, a plurality of devices 25 (shown in FIG. 3) in communication with the control panel 22, and a plurality of sensors 26 (shown in FIG. 2) configured to monitor the conditions of the sleeping chamber 14. A plurality of speakers 36 may be mounted on the walls 16 of the main structure 12. In one form of the present disclosure, the plurality of speakers may be mounted at upper portions of the walls 16. Optional components of the sleep suite 10 may also include, by way of example, appliances such as a refrigerator, microwave, or coffee maker, among others. Referring to FIG. 2, the plurality of sensors 26 may be mounted on one or more walls 16 of the main structure 12 throughout the sleep suite 10 or outside the sleep suite 10 and in communication with the control panel 22, the plurality of sensors 26 configured to continuously monitor the conditions of the sleeping chamber 14. The plurality of sensors 26 may be arranged in an array.

Referring back to FIG. 1, the mattress or the sleeping surface 18 may be a typical mattress, a customized mattress specific for the sleep suite 10, or a smart mattress with sensors inside. The blanket and pillow set is provided in the sleep suite 10 and may include a bed sheet, pillows and a blanket. The bed sheet, pillows, and blanket may be cohesive or customizable, may or may not be utilizing sensors (e.g., “smart” sheets, pillow, blankets), may be organic, self-cleaning, and/or may have transparent properties.

The cabinet 20 may include a plurality of customizable compartments including a medicine cabinet and a compartment for a mini fridge. The control panel 22 is mounted on a surface of the cabinet 20. The control panel 22 may be activated by voice, passcodes, or key FOB, among others. The foot board 24 may provide an additional storage space. Further, the features as set forth herein may be activated by gesture control.

Referring to FIG. 3, the plurality of devices 25 that may be installed in the sleep suite 10 include, but are not limited to, multiple noise cancellation devices 30, a lighting device 32, an aromatherapy device 34, a plurality of speakers 36 (also shown in FIG. 1), a fan 38, a chromotherapy device 40, a humidifier 42, a dehumidifier 44, an air filter 46, a ventilation device 48, a water filter 50, a computer 52, and a hard drive 54. The multiple noise cancelling devices 30 may be mounted inside or outside the sleep suite 10. Other devices may include a water tank and inlet, along with batteries to power the various devices.

The lighting device 32 may include at least one strip LED lighting provided in the frame of the main structure 12 and may be controlled as a whole or in zones. Other forms of lighting may also be employed and thus the use of LED lighting is merely exemplary and should not be construed as limiting the scope of the present disclosure. The computer 52 may be provided under the mattress, in a headboard 29 (shown in FIG. 1), or at any location within or near the sleep suite 10 and its various components, and may be provided to process and/or analyze some or all of data received from the plurality of sensors 26. The computer 52 may include a sleep tracker reporting app and an interface for full data display. In addition to the computer 52, some of the other devices may also be disposed under the mattress 18, including but not limited to, the humidifier, the water tank for the humidifier/dehumidifier, the hard drive, the air filter, and the water filters.

The control panel 22 includes a thumb print reader 56, a face reader 58, and a connection interface 60 for data synchronization of a hand-held device (not shown), such as a smart phone, tablet, smart watch, or other portable electronic device, whether a wearable or non-wearable. The plurality of devices 25 may be connected to the control panel 22, through wireless communications or through wire connections. The connection interface 60 allows a user to control the plurality of devices 25 simultaneously by connecting a hand-held device to the connection interface 60. The hand-held device may store the settings for some or all of the plurality of devices 25. In one form, the settings for the plurality of devices 25 may be saved in a phone application of the hand-held device. When the hand-held device is plugged into another sleep suite, or wirelessly connected to another sleep suite, the settings saved in the phone application may be synchronized such that one or more of the plurality of devices 25 can be automatically turned on and adjusted according to the saved settings without the need to turn on and adjust the plurality of devices 25 individually.

Alternatively, the control panel 22 may include a plurality of control buttons or knobs corresponding to the plurality of devices 25, the plurality of control buttons or knobs configured to control the plurality of devices 25 individually. Alternatively, the control panel 22 may be activated by voice, passcodes, or key FOB, among others. For example, the voice activated commands may be received by a voice recognition software of the control panel 22 to carry out control of one or more of the plurality of devices 25 in the sleep suite 10. Alternatively, instead of using a hand-held device, the control panel 22 may be configured to access data relating to settings for the plurality of devices 25 from a cloud server.

As further shown in FIG. 3, the plurality of sensors 26 may be mounted on one or more walls 16 of the main structure 12 throughout the suite or outside the sleep suite 10, the plurality of sensors 26 configured to continuously monitor the conditions of the sleeping chamber 14. The plurality of sensors 26 include, but are not limited to, a temperature sensor 62, a humidity/moisture sensor 64 configured to detect the water/sweat content in the air, one or more audio sensors 66 (including an internal audio sensor and an external audio sensor) configured to detect noises inside and external of the sleep suite 10, an aroma sensor 68 configured to measure aroma ppm levels and the odor, one or more motion sensors 70 (including an internal motion sensor and an external motion sensor) configured to detect motion inside and outside the sleep suite 10, an infrared sensor 72, an insect detector 74, a weight/pressure sensor 76 installed on bed and configured to detect the weight on bed, a dust sensor 78 configured to detect bacteria/allergen/dust inside the sleep suite 10, one or more light sensors 80 (including an internal light sensor and an external light sensor) configured to detect brightness inside and outside the sleep suite 10, a gas sensor 82 configured to detect the level of oxygen and CO₂ or any gases besides oxygen and CO₂, and a brainwave sensor 84 configured to detect brainwaves. Any one of the plurality of sensors 26 may be configured to work with other devices inside the sleep suite 10 to form Internet of Things (IOT) in the sleep suite 10.

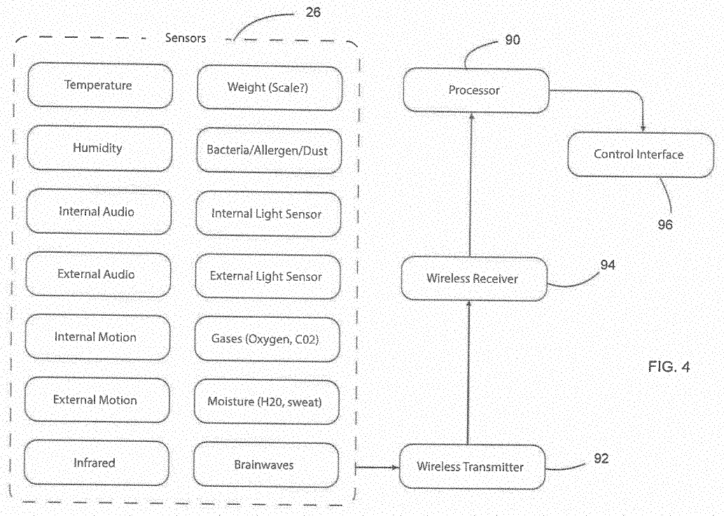

Referring to FIG. 4, the plurality of sensors 26 may transmit signals relating to various measurements of the conditions of the sleep suite 10 to a processor 90 through a wireless transmitter 92 and a wireless receiver 94. More specifically, the plurality of sensors 26 may be connected to a wireless transmitter 92, which in turn transmits signals relating to various measurements of the conditions of the sleep suite 10 by the sensors 26 to a wireless receiver 94 on the side of the control panel 22. The wireless receiver 94 receives the signals and sends the signals to a processor 90. The processor 90 may process the received signals, compare the various measurements by the sensors 26 against the stored predetermined or desired settings, determine a desired change of operation of at least one of the plurality of devices 25, and send a command regarding the desired change of operation to a control interface 96. The control interface 96 is installed in the control panel 22 and is configured to control the operation of the plurality of devices 25. The processor 90 may be installed in the control panel 22, or may be located in a network server or a cloud server.

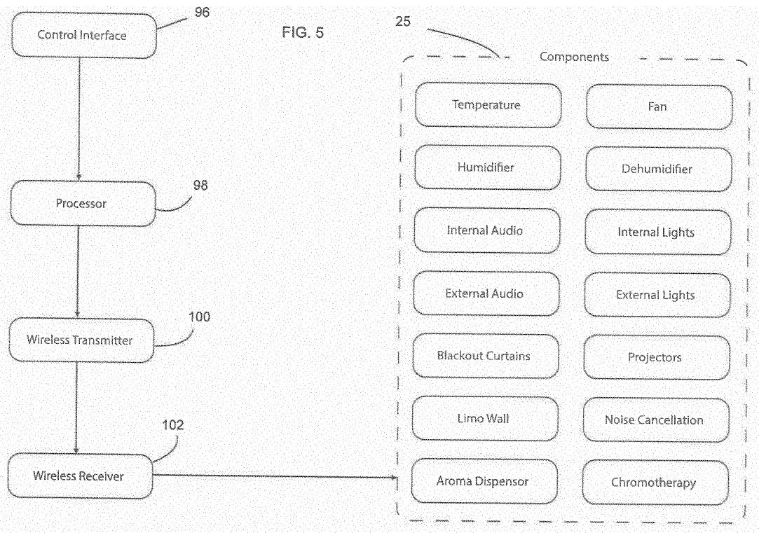

Referring to FIG. 5, after receiving the commands from the processor 90 regarding the determined change of operation of at least one of the plurality of devices 25, the control interface 96 may send the determined command to another processor 98, which converts the determined command into appropriate signals that can be sent by another wireless transmitter 100 to another wireless receiver 102 connected to the plurality of devices 25. The operation of one or more of the devices 25 may be adjusted accordingly.

The sleep suite 10 may define different zones. Data from the sensors 26 may be used for one or more than one purpose. The data acquired by the plurality of sensors 26 may be stored in the sleep suite 10 as raw data or may be transmitted to network for further analysis and/or processing. The data acquired by the plurality of sensors 26 may be transmitted to an external device (such as the processor 90 shown in FIG. 4) through the control panel 22 for analysis for determining the comfort level inside the sleep suite 10. The external device may transmit a signal back to the control panel 22 suggesting preferred settings for the plurality of devices 25 or automatically control the plurality of devices 25 accordingly to achieve an optimum comfort level for the user. In another variation, an external control center (not shown) may be employed to monitor and control the sleep suite 10 remotely. The data and/or process data may be stored in the cloud.

The sleep suite 10 constructed in accordance with the teachings of the present disclosure can automatically and simultaneously control the plurality of devices in the sleep suite according to the predetermined settings saved in a hand-held device, a computer, a network server, or a cloud server. Therefore, the plurality of devices are controlled and adjusted to provide an optimum comfort level tailored for a particular user. In addition to providing a place to sleep, the sleep suite 10 can be used to provide aromatherapy, chromotherapy, meditation, intimacy, or recovery, among other treatments/environments.

The description of the disclosure is merely exemplary in nature and, thus, variations that do not depart from the substance of the disclosure are intended to be within the scope of the disclosure. For example, it should be understood that the sleep suite 10 may be configured as set forth herein for human use, canine or feline use, or use by any subject for which improved sleeping conditions are desired. Such variations are not to be regarded as a departure from the spirit and scope of the disclosure.

What is claimed is:

1. A sleep suite comprising:
a main structure defining a sleep chamber;
a plurality of sensors configured to monitor conditions of the sleep chamber;
a plurality of devices configured to change the conditions of the sleep chamber; and
a control panel configured to automatically control the plurality of devices to change the conditions of the sleep chamber based on predetermined settings for the plurality of devices and the conditions monitored by the plurality of sensors.

2. The sleep suite according to claim 1, wherein the plurality of devices include at least one of a noise cancellation device, a lighting device, an aromatherapy device, a speaker, a fan, a chromotherapy device, a humidifier, a dehumidifier, an air filter, a ventilation device, and a water filter.

3. The sleep suite according to claim 1, wherein the plurality of sensors include at least one of temperature sensor, a humidity sensor, an audio sensor, an aroma sensor, a motion sensor, an infrared sensor, an insect detector, a weight sensor, a dust sensor, a light sensor, a gas sensor, and a brainwave sensor.

4. The sleep suite according to claim 1, further comprising a computer connected to the plurality of sensors, the computer configured to process and analyze information acquired by the plurality of sensors and related to the conditions of the sleep chamber.

5. The sleep suite according to claim 1, further comprising a hand-held device configured to be connected to a connection interface of the control panel, wherein the hand-held device stores the predetermined settings for the plurality of devices.

6. The sleep suite according to claim 5, wherein the control panel controls the plurality of devices simultaneously according to the predetermined settings stored in the hand-held device when the hand-held device is connected to the connection interface.

7. The sleep suite according to claim 1, wherein the control panel further includes at least one of a thumb print reader and a face reader.

8. The sleep suite according to claim 1, wherein the control panel is configured to be activated by at least one of voice, passcodes, or key FOB.

9. The sleep suite according to claim 1, wherein the control panel is configured to access data relating to the predetermined settings of the plurality of devices from a computer, a hand-held device, a network server, or a cloud server.

10. The sleep suite according to claim 1, wherein the plurality of sensors are arranged in an array throughout the sleep suite.

11. The sleep suite according to claim 10, further comprising at least one wireless transmitter connected to the plurality of sensors, the at least one wireless transmitter configured to transmit signals relating to measurements of the conditions of the sleep suite to a wireless receiver of the control panel.

12. The sleep suite according to claim 11, wherein the wireless receiver transmits the signals to a processor configured to determine a desired change of operation of at least one of the plurality of devices.

13. The sleep suite according to claim 12, wherein the processor is located in a computer in the sleep suite, a network server, or a cloud server.

14. The sleep suite according to claim 12, further comprising a wireless receiver connected to the plurality of devices, the wireless receiver configured to receive a command from the processor relating to the desired change of operation for the at least one of the plurality of devices.

15. The sleep suite according to claim 1, wherein the predetermined settings are stored in a computer, a network server, a cloud server, or a hand-held device.
